(19) United States
(12) Patent Application Publication
(10) Pub. No.: US 2008/0139609 A1
(43) Pub. Date: Jun. 12, 2008

(54) GRANISETRON COMPOSITIONS

(76) Inventors: Bikash Kumar Sahoo, Cuttack (IN); Hiren Patel, Ahmedabad (IN); Srinivas Irukulla, Hyderabad (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/936,140

(22) Filed: Nov. 7, 2007

Related U.S. Application Data
(60) Provisional application No. 60/891,257, filed on Feb. 23, 2007.

(30) Foreign Application Priority Data
Nov. 8, 2006 (IN) 2056/CHE/2006

(51) Int. Cl. A61K 31/439

(52) U.S. Cl. 514/304

(57) ABSTRACT

Stable liquid pharmaceutical compositions comprise granisetron or a pharmaceutically acceptable salt thereof.

### INTRODUCTION TO THE INVENTION

The present invention relates to pharmaceutical compositions comprising granisetron or its pharmaceutically acceptable salts, solvates, enantiomers or mixtures thereof, processes for preparing such compositions, and their methods of use. An aspect of the present invention relates to stable liquid pharmaceutical compositions comprising granisetron or its salts, for oral or parenteral administration.

Granisetron is a selective 5-hydroxytryptamine₃ (5-HT₃) receptor antagonist with little or no affinity for other serotonin receptors. Granisetron hydrochloride (the active ingredient in KYTRIL® injection, KYTRIL® oral solution and KYTRIL® tablets, sold by Roche Laboratories Inc., USA) is a white to off-white solid that is readily soluble in water and in normal saline at 20° C., and has a chemical name endo-N-(9-methyl-9-azabicyclo[3.3.1]non-3-yl)-1-methyl-1H-indazole-3-carboxamide hydrochloride with a molecular weight of 348.9 (312.4 for the free base). It is structurally represented by Formula I.

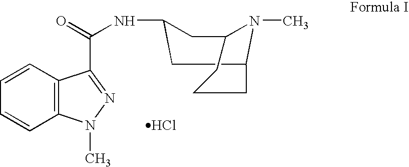

KYTRIL® Injection is a clear, colorless, sterile, nonpyrogenic, aqueous solution for intravenous administration. KYTRIL® Injection 1 mg/1 mL is available in 1 mL single-use vials and 4 mL multi-use vials with benzyl alcohol as the preservative. KYTRIL® Injection 0.1 mg/1 mL is available in 1 mL single-use vials with no preservative. The strengths are expressed as contained granisetron.

KYTRIL® injection and KYTRIL® oral solution are indicated for the prevention of nausea and/or vomiting associated with initial and repeat courses of emetogenic cancer therapy. In addition, KYTRIL® injection is also indicated for prevention and treatment of postoperative nausea and vomiting.

U.S. Pat. No. 6,294,548 discloses a parenteral formulation comprising granisetron hydrochloride, and contains benzyl alcohol as a preservative. This patent teaches against the use of methyl paraben or propyl paraben or their combinations, since these preservatives were found to degrade during sterilization by autoclaving. The patent also describes unsuitability of meta-cresol as a preservative because of its incompatibility with granisetron hydrochloride.

International Application Publication No. WO 2007/069070 discloses a multidose injectable dosage form that includes granisetron or a salt and one or more preservatives, wherein the preservative is other than benzyl alcohol. The process for preparing the compositions includes aseptically filling the compositions into multidose containers, wherein a sterilizing grade membrane used in the process imparts sterility.

Surprisingly, it was found that liquid pharmaceutical compositions of the present invention, comprising granisetron hydrochloride are adequately stabilized by use of parabens including methyl paraben or propyl paraben or their combinations when a sterilization process using an autoclave is employed.

Thus, the present invention provides for an alternative liquid pharmaceutical compositions comprising granisetron with desired stability for oral or parenteral administration.

### SUMMARY OF THE INVENTION

An aspect of the invention provides stable liquid pharmaceutical compositions comprising granisetron or its pharmaceutically acceptable salts, and a preservative other than benzyl alcohol.

Another aspect of the invention provides stable liquid pharmaceutical compositions comprising granisetron or its pharmaceutically acceptable salts, and a preservative other than benzyl alcohol, for oral or parenteral administration.

In one embodiment of the invention, stable liquid pharmaceutical compositions for parenteral administration comprise granisetron hydrochloride and, as a preservative, a combination of methyl paraben and propyl paraben.

In another embodiment of the invention, stable pharmaceutical compositions for parenteral administration comprise granisetron hydrochloride, methyl paraben, and propyl paraben in an aqueous vehicle, optionally with a buffering agent.

In another embodiment, a preservative content of the composition of the present invention ranges from about 0.01% w/w to about 1% w/w, or about 0.05% to about 0.5% w/w, or about 0.1% w/w to about 0.3% w/w.

In one embodiment, liquid pharmaceutical compositions of the present invention include methyl paraben in the range of from about 0.01% to about 1%, or from about 0.05% to about 0.5% w/w of the total composition.

In one embodiment, liquid pharmaceutical compositions of the present invention include parabens such as propyl paraben in the range of from about 0.01% to about 0.5%, or from about 0.01% to about 0.25%, w/w of the total composition.

In one embodiment, liquid pharmaceutical compositions of the present invention include ratios of methyl paraben to that of propyl paraben in the range of about 25:1 to 1:1, or about 15:1 to 5:1.

In an embodiment the invention includes stable liquid compositions comprising granisetron or its salts wherein the total drug-related impurities of the composition are not more than 2% of the granisetron content.

In an embodiment the invention includes stable liquid compositions comprising granisetron or its salts having a 4-hydroxybenzoic acid content not more than or about 0.4% w/w, or not more than or about 0.1% w/w.

In another embodiment of the invention, pharmaceutical compositions comprising granisetron or its salts are filled into containers and the containers are subjected to terminal sterilization.

Another embodiment of the present invention provides solid oral pharmaceutical compositions comprising granisetron hydrochloride.

In embodiments, the invention includes processes for the preparing the pharmaceutical compositions and methods of using such compositions.

An embodiment of the invention provides an aqueous pharmaceutical composition comprising granisetron or a salt thereof and one or more paraben preservatives, the composition being sterilized by exposure to heat.

Another embodiment of the invention provides an aqueous pharmaceutical composition comprising a salt of granisetron and about 0.01 percent to about 1 percent by weight of one or more paraben preservatives, the composition being sterilized by exposure to heat.

A further embodiment of the invention provides an aqueous pharmaceutical composition comprising a salt of granisetron and about 0.01 percent to about 1 percent by weight of a mixture of methyl paraben and propyl paraben, the composition being sterilized by exposure to heat and having a 4-hydroxybenzoic acid content not more than about 0.1 percent by weight.

### DETAILED DESCRIPTION OF THE INVENTION

The present invention provides stable liquid pharmaceutical compositions comprising granisetron or its pharmaceutically acceptable salts, and a preservative other than benzyl alcohol.

Liquid pharmaceutical compositions, in the context of the present invention, comprise solutions, dispersions such as emulsions and suspensions, syrups, dry suspensions/syrups to be reconstituted before use, and the like and mixtures thereof. Non-limiting examples of liquid vehicles used in such compositions include water, alcohols, oils, and the like and mixtures thereof, which are suitable for oral or parenteral administration.

In one embodiment of present invention, liquid pharmaceutical compositions comprising granisetron or its salts are intended for oral or parenteral administration.

In another embodiment, liquid pharmaceutical compositions of the present invention comprising granisetron hydrochloride are useful for parenteral administration.

In the context of the invention, liquid pharmaceutical compositions comprising granisetron or its salts further contain preservatives other than benzyl alcohol, that have been found to be stable and compatible.

Non-limiting examples of preservatives that can be employed in the context of the invention include parabens such as methyl paraben and propyl paraben, meta-cresol, para-cresol, bronopol, benzalkonium chloride, and the like and mixtures thereof.

In one embodiment, liquid pharmaceutical compositions of the present invention are stable, in terms of degradation of contained preservatives and formation of impurities, when stored at accelerated conditions of temperature and humidity, such as at 40° C. and 75% relative humidity (“RH”). Impurities, which may be originally present or formed during storage, can include:

1) 2-Methyl-N (1R,3r,5S)-9-methyl-9-azabicyclo[3.3.1]non-3-yl]-2H-indazole-3-carboxamide (“Impurity A”), a process impurity and is not formed during the shelf life of the product, which is represented by Formula II;

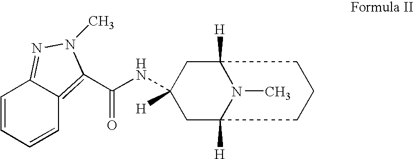

2) N-(1R,3r,5S)-9-methyl-9-azabicyclo[3.3.1]non-3-yl]-1H-indazole-3-carboxamide (“Impurity B”), a process impurity formed by oxidative degradation and represented by Formula III;

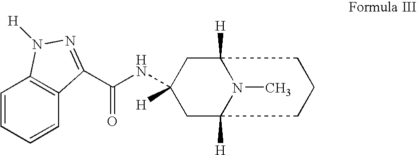

3) N-(1R,3r,5S)-9-azabicyclo[3.3.1]non-3-yl]-1-methyl-1H-indazole-3-carboxamide (“Impurity C”), formed by oxidative degradation and represented by Formula IV;

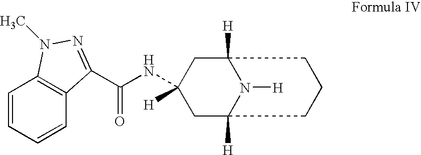

4) 1-Methyl-1H-indazole-3-carboxylic acid (“Impurity D”), a starting material and degradant which is formed in highly acidic conditions and under UV light exposure, and is represented by Formula V;

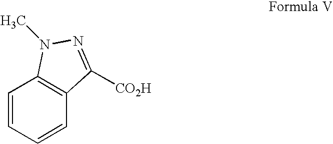

5) (1R,3r,5S)-9-methyl-9-azabicyclo[3.3.1]nonan-3-amine (“Impurity E”), which is represented by Formula VI; and

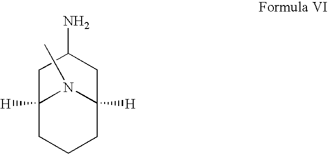

6) 4-hydroxybenzoic acid, which a degradation product of methylparaben and propylparaben and is represented by Formula VIII.

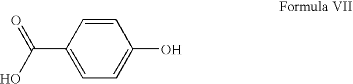

The 4-Hydroxybenzoic acid impurity content can be analyzed using a high performance liquid chromatography procedure, such as that described below.

Buffer Preparation:

Step 1) 1.6 ml of orthophosphoric acid is dissolved in 880 mL of purified (such as Milli-Q) water.

Step 2) 1 ml of hexylamine is added to the above solution and mixed well, then the pH is adjusted to 7.3±0.02 with triethylamine.

Mobile phase A: Mix buffer, acetonitrile and methanol in the ratio of 850:60:90 v/v respectively and degas in a sonicator.

Mobile phase B: Mix buffer, acetonitrile and methanol in the ratio of 10:80:10 v/v respectively and degas in a sonicator

With the above mobile phases, a gradient program of 0, 15, 55, 57, 65 minutes is performed with varying compositions of mobile phase for each time period, as given in the table below:

| Time | | |
|---|---|---|
| (minutes) | % of Mobile Phase A | % of Mobile Phase B |
| 0 | 100 | 0 |
| 15 | 100 | 0 |
| 55 | 70 | 30 |
| 57 | 100 | 0 |
| 65 | 100 | 0 |

Diluent: Purified (Milli-Q) water.

Chromatographic system: Liquid chromatograph equipped with a 305 nm UV-visible detector.

Column: ACE, C18, 150 mm×4.6 mm, 5 μm.

Column temperature: 40° C.

Flow rate: 1.5 mL/minute.

Injection volume: 20 μL of 0.1 mg/mL granisetron solution. Solutions of higher strength will be diluted volumetrically to this concentration.

Limit of detection (LOD) for 4-OH benzoic acid: less than 0.01% of the total composition.

Limit of quantification (LOQ) for 4-OH benzoic acid: 0.01% of the total composition. Retention time (RT): 1.82 minutes.

Relative retention time (RRT): 0.05 (where granisetron is assigned a value of 1.)

As is known to those skilled in the art, particular instruments will use different conditions and give different retention times and limits of detection and quantification; the above information is provided to exemplify conditions with a typical instrument.

In an embodiment, the invention includes stable liquid compositions comprising granisetron or its salts wherein the compositions comprise less than about 0.5%, or less than about 0.3%, by weight of 4-hydroxybenzoic acid.

In an embodiment, a preservative content of the composition of the present invention ranges from about 0.01% w/w to about 1% w/w, or about 0.05% w/w to about 0.5% w/w, or about 0.1% w/w to about 0.3% w/w.

In one embodiment, liquid pharmaceutical compositions of the present invention include methyl paraben in the range of from about 0.01% to about 1%, or from about 0.05 to about 0.5% w/w of the total composition.

In one embodiment, liquid pharmaceutical compositions of the present invention include parabens such as propyl paraben in the range of from about 0.01% to about 0.5% w/w, or from about 0.01% to about 0.25% w/w, of the total composition.

In one embodiment, liquid pharmaceutical compositions of the present invention include weight ratios of methyl paraben to propyl paraben in the range of about 25:1 to 1:1, or about 15:1 to 5:1.

In embodiments of the invention, compositions comprising granisetron further optionally comprise one or more pharmaceutically acceptable excipients for parenteral use, such as but not limited to stabilizers, antioxidants, pH modifiers, isotonicity adjusters and such others, as known to a person skilled in the art.

Pharmaceutically acceptable antioxidants include, without limitation, natural vitamin E, vitamin-E succinate, ascorbic acid, sodium metabisulfite, aminoacids, flavones, monothioglycerol, L-cysteine, thioglycolic acid, and mixtures thereof. Such antioxidants may be used in the concentration ranges of about 0.1% to about 15% w/w, or about 0.5% to about 5% w/w.

Non-limiting examples of pH modifiers and stabilizers include citric acid, tartaric acid, succinic acid, glutamic acid, ascorbic acid, lactic acid, acetic acid, malic acid, maleic acid, and sodium salts thereof, sodium hydroxide, sodium carbonate, sodium bicarbonate, tris buffer, meglumine, amino acids, and mixtures thereof. Such pH modifiers and stabilizers are included to maintain pH between about 2 and about 7, or between about 4 and about 6, in the composition.

Non-limiting examples of isotonicity modifiers include sodium chloride, dextrose, mannitol, lactose, propylene glycol, and glycerine. Such isotonicifiers are used to provide an osmolality between about 280 and about 320 mOsm/kg.

In an embodiment, a process for preparation of composition of the present invention comprises:

1. Dissolving preservatives, and optionally other pharmaceutical additives, in water for injection,

2. dissolving granisetron hydrochloride in the above solution,

3. aseptically filtering and filling the solution of step 2 into glass vials, and

4. terminally sterilizing the sealed vials by autoclaving.

The stable liquid pharmaceutical compositions comprising granisetron or its salts of the present invention can be packed in single-dose or multi-dose vials for parenteral administration, or in bottles suitable for oral use.

In embodiments, the pharmaceutical compositions and formulations of the present invention are administered to patients in need thereof through the parenteral route.

If the compositions or pharmaceutical formulations are to be administered parenterally, they must be rendered sterile prior to such administration. Any of the several known means for rendering such pharmaceutical preparations sterile may be used so long as the drug is not inactivated.

Sterilization plays an important role for parenterals. It is defined as any process that effectively kills or eliminates transmissible agents such as fungi, bacteria, viruses, prions and spore parenterals, and it is achieved by different types of sterilization such as but not limited to moist heat sterilization (autoclaving), filtration, radiation, gaseous sterilization and the like. Various sterilization techniques are discussed below.

### Moist Heat Sterilization:

Autoclaves commonly use steam heated to 121° C. (250° F.), at 103 kPa (15 psi) pressure. Solid surfaces are effectively sterilized when heated at this temperature for at least 15 minutes, or to 134° C. for a minimum of 3 minutes. After sterilization, autoclaved liquids must be cooled slowly to avoid boiling when the pressure is released.

### Radiation Sterilization:

Methods exist to sterilize using radiation such as X-rays, gamma rays, or subatomic particles:

a) Gamma rays are very penetrating and are commonly used for sterilization of disposable medical equipment, such as syringes, needles, cannulas and IV sets. Gamma radiation requires bulky shielding for the safety of the operators; they also require storage of a radioisotope (such as cobalt-60), which continuously emits gamma rays.

b) Ultraviolet light irradiation (UV, from a germicidal lamp) is useful for sterilization of surfaces and some transparent objects.

### Filtration:

Clear liquids that would be damaged by heat, irradiation or chemical sterilization can be sterilized by mechanical filtration. This method is commonly used for sensitive pharmaceuticals and protein solutions in biological research. A filter with pore sizes of 0.2 μm will effectively remove bacteria. If viruses must also be removed, a much smaller pore size about 20 nm is needed. Solutions filter slowly through membranes with smaller pore diameters. The filtration equipment and the filters themselves may be purchased as presterilized disposable units in sealed packaging, or must be sterilized by the user, generally by autoclaving at a temperature that does not damage the fragile filter membranes. To ensure sterility, the filtration system must be tested to ensure that the membranes have not been punctured prior to or during use.

To ensure the best results, pharmaceutical sterile filtration is performed in a room with highly filtered air (HEPA filtration) or in a laminar flow cabinet or “flowbox,” a device which produces a laminar stream of HEPA filtered air.

In an embodiment of the invention, the moist heat sterilization (autoclaving) technique has been found to be particularly useful. After sterilization, the composition is filled into a sterile container and the container is sealed.

The facility used for processing the liquid compositions and formulations of the present invention should comply with requirements given in the current edition of the United States Pharmacopoeia, published by United States Pharmacopeial Convention, Inc., Rockville, Md. for parenteral dosage forms.

In an embodiment, the invention includes stable liquid compositions comprising granisetron or its salts.

The stable liquid pharmaceutical compositions comprising granisetron or its salts of the present invention can be used for oral or parenteral administration for the prevention of nausea and/or vomiting associated with initial and repeat courses of emetogenic cancer therapy, prevention and treatment of postoperative nausea, vomiting, migraine, trigeminal neuralgia, anxiety and psychosis.

In an embodiment the invention includes solid oral pharmaceutical compositions comprising granisetron or its salts and at least one pharmaceutical excipient.

The solid oral pharmaceutical compositions of the present invention includes granisetron or a pharmaceutically acceptable salt and at least one pharmaceutically acceptable excipient. Pharmaceutically acceptable excipients include but are not limited to diluents, binders, lubricants, film forming agents, plasticizers, colorants and the like.

### Diluents:

Various useful diluents include but are not limited to starches, lactose, mannitol, cellulose derivatives and the like. Different grades of lactose include but are not limited to lactose monohydrate, lactose DT (direct tableting), lactose anhydrous, Flowlac™ (available from Meggle Products), Pharmatose™ (available from DMV) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, tribasic calcium phosphate, maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others.

### Binders:

Various useful binders include but are not limited to hydroxypropylcellulose (Klucel™ LF), (Klucel EXF), hydroxypropyl methylcellulose or hypromellose 5 cps (Methocel E5 Premium™), polyvinylpyrrolidone or povidone (PVP-K25, PVP-K29, PVP-K30, PVP-K90), Plasdone™ S 630 (copovidone), powdered acacia, gelatin, guar gum, carbomer (e.g. carbopol), methylcellulose, polymethacrylates, and starch.

### Lubricants:

An effective amount of any generally accepted pharmaceutical tableting lubricant can be added to assist with compressing into tablets. Useful tablet lubricants include but are not limited to magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Film-Forming Agents:

Various useful film forming agents include but are not limited to cellulose derivatives such as soluble alkyl- or hydroalkylcellulose derivatives such as methyl cellulose, hydroxymethyl cellulose, hydroxyethyl cellulose, hydroxypropyl cellulose, hydroxymethyethyl cellulose, hydroxypropyl methylcellulose, sodium carboxymethyl cellulose, etc., acidic cellulose derivatives such as cellulose acetate phthalate, cellulose acetate trimellitate and methylhydroxypropylcellulose phthalate, polyvinyl acetate phthalate, etc., insoluble cellulose derivatives such as ethyl cellulose and the like, dextrins, starches and starch derivatives, polymers based on carbohydrates and derivatives thereof, natural gums such as gum Arabic, xanthans, alginates, polyacrylic acid, polyvinyl alcohol, polyvinyl acetate, polyvinylpyrrolidone, polymethacrylates such as derivatives thereof (Eudragit™), chitosan and derivatives thereof, shellac and derivatives thereof, waxes and fat substances.

As an alternative to the above coating ingredients, ready-mix coating materials, such as, for example, OPADRY White YS-1-7003, may be employed. OPADRY White YS-1-7003 contains titanium dioxide, HPMC 3 cPs, HPMC 6 cPs, Macrogol/PEG400 and polysorbate, and is supplied by Colorcon. Pre-formulated coating products are convenient, requiring only combining with a liquid prior to use.

### Plasticizers:

Various plasticizers for coatings include but are not limited to castor oil, diacetylated monoglycerides, dibutyl sebacate, diethyl phthalate, glycerin, polyethylene glycol, propylene glycol, triacetin, and triethyl citrate. Also mixtures of plasticizers may be utilized. The type of plasticizer depends upon the type of coating agent. A plasticizer is normally present in an amount ranging from 5% (w/w) to 30% (w/w) based on the total weight of a film coating.

An opacifier like titanium dioxide may also be present in an amount ranging from about 10% (w/w) to about 20% (w/w) based on the total weight of the coating. When colored tablets are desired then the colour is normally applied in the coating. Consequently, colouring agents and pigments may be present in the film coating. Various colouring agents include but are not limited to ferric oxides, which can either be red, yellow, black or blends thereof.

### Containers:

The compositions may be provided in suitable single dose containers by aseptically filing suitable containers with the sterile solution and then hermetically sealing the single dose container or the compositions may be provided in suitable multidose containers, wherein the composition is filled into the containers and then subjected to sterilization.

By “suitable containers” is meant containers capable of maintaining a sterile environment, such as a vial capable of being hermetically sealed by a stopper means. Additionally, suitable containers imply appropriateness of size, considering the volume of solution to be held upon reconstitution of the lyophilized composition, and appropriateness of the container material, frequently USP Type I glass. The stopper means employed, e.g. sterile rubber closures or an equivalent should be understood to be that which provides the aforementioned seal but which also allows entry for the purpose of introduction of diluent, e.g. sterile water. These and other aspects of the suitability of containers for pharmaceutical products such as those of the instant invention are well known to those skilled in the practice of the pharmaceutical arts.

Liquid compositions of the present invention for parenteral use should passes USP Test 788 for Particulate Matter in Injections. The USP particulate matter test defines the numbers of foreign particulate matter as observed by optical microscopy. In USP Test 788, the limit for foreign particulate matter having sizes greater than or equal to 10 microns is 3000 per container, and for particles having size greater than or equal to 25 microns is 300 per container.

In another embodiment, the invention includes the use of packaging materials such as containers including lids, composed of polyethylene and or polypropylene and/or glass, and blisters or strips composed of aluminum, high-density polypropylene, polyvinyl chloride, or polyvinyl chloride coated with polyvinylidene dichloride, the latter generally called PVC/PVDC. Different grades of PVC/PVDC are available as PVC/PVDC 40 gsm, PVC/PVDC 60 gsm, PVC/PVDC 90 gsm, PVC/PVDC 120 gsm etc. PVC/PVDC 40 gsm means 40 grams of PVDC coating per square meter of PVC foil. Similarly 60 gsm means 60 grams of PVDC coating per square meter of PVC foil, 90 gsm means 90 grams of PVDC coating per square meter of PVC foil, etc.

The content of granisetron and its impurities in solutions can be determined by techniques such as high performance liquid chromatography.

Solid dosage forms can be tested to determine the rate and extent of drug that is released upon immersion in aqueous media, such as using Test 711 “Dissolution” in United States Pharmacopeia 29, United States Pharmacopeial Convention, Inc., Rockville, Md., 2005 (“USP”).

The following examples are provided solely to further illustrate certain specific aspects and embodiments of the invention, and are not intended to limit the scope of the invention.

### EXAMPLES 1-2

### Granisetron Hydrochloride (1 mg/mL) Injection Compositions

| | Grams | |
|---|---|---|
| | Example 1 | Example 2 |
| | (Single-dose | (Multi-dose |
| Ingredient | package) | package) |
| Granisetron hydrochloride | 1.12 | 1.12 |
| Citric acid anhydrous | 2 | 2 |
| Sodium chloride | 9 | 9 |
| Methyl paraben | — | 1.8 |
| Propyl paraben | — | 0.2 |
| Sodium hydroxide | q.s. to pH 5 ± 0.1 | q.s. to pH 5 ± 0.1 |
| (1N aqueous solution) | | |
| Hydrochloric acid | q.s. to pH 5 ± 0.1 | q.s. to pH 5 ± 0.1 |
| (1N aqueous solution) | | |
| Water for injection | q.s. to 1 liter | q.s. to 1 liter |

Manufacturing Process:

1. Water for injection (about 750 mL) was placed into a vessel and (for Example 2 only) was heated to 50-60° C.

2. Methyl paraben and propyl paraben were dissolved in the step 1 water for injection (Example 2 only) and the solution cooled to room temperature.

3. Citric acid and sodium chloride were dissolved in the water of step 1 or the solution of step 2.

4. The pH of solution of step 3 was adjusted to 5±0.1 using sodium hydroxide (1N aqueous solution) or hydrochloric acid (1N aqueous solution).

5. Granisetron hydrochloride was dissolved in the solution of step 4 with stirring and the volume was made up to 1 liter with water for injection.

6. Solution of step 5 was aseptically filtered through a PVDF filter having pore sizes 0.22 μm.

7. The filtrate of step 6 was aseptically filled into USP type 1 glass vials (fill volume of 1 mL for Example 1, and fill volume of 4 mL for Example 4), and the vials were stoppered and sealed.

8. The vials from step 7 were terminally sterilized by autoclaving at 121° C. for 20 minutes.

Alternatively, the above vials of step 7 may be subjected to terminal sterilization by radiation exposure or by gaseous sterilization by using ethylene oxide. Brief representative processes for sterilization by these two techniques are described below:

Sterilization by Radiation Exposure:

1. Load finished product containers into carriers.

2. Raise a radiation source from a shielding pool to the irradiation position.

3. The position of source is monitored throughout the cycle.

4. Carriers are moved with the help of a conveyor into a containment structure along a track.

5. Carriers are moved about the radiation source along a pathway defined by the track at a sufficient index speed to obtain an effective radiation dose. Speed is controlled to produce the required dose.

6. The carriers exit the containment structure on the track and are moved to an unloading station and product containers are removed from the carriers.

7. Source is lowered into the shielding pool.

| | Specific |
|---|---|
| Parameters | Parameters |
| Radiation Source | ⁶⁰CO |
| Radiation Dose | about 10 to 15 KGy |

Gaseous Sterilization:

1. Load the samples in a gas-tight ethylene oxide exposure chamber and apply vacuum to remove air (to prevent a unsafe mixture when ethylene oxide is introduced). Up to 97% air is to be removed. This can be done by repeated application of vacuum and nitrogen about 3 to 5 times.

2. Introduce steam to replace the moisture lost during vacuum application.

3. Introduce ethylene oxide gas to assure adequate sterilization.

4. Maintain the product in ethylene oxide for a predetermined time.

5. Sequentially apply vacuum and nitrogen repeatedly about 3 to 5 times until chamber atmosphere is clean and material safe to remove from the chamber.

| Parameters | Specific Parameters |
|---|---|
| Ethylene oxide used | about 400 mg/L to 700 mg/L of chamber volume |
| Dwell time | About 2.5 hours |

### EXAMPLE 3

### Stability Study of the Composition of Example 2

Samples were treated, as described in the following table, then the preservative and preservative decomposition product contents were analyzed.

| | | Propyl | |
|---|---|---|---|
| | Methyl Paraben | Paraben | 4-Hydroxybenzoic |
| Sample | (% w/w)* | (% w/w)* | Acid (% w/w)** |
| Before autoclaving | 102.8 | 100.7 | Not detected |
| After autoclaving 20 | 101.4 | 109.8 | Not detected |
| minutes at 121° C. | | | |
| Stored 1 month at | 100.5 | 108 | 0.0401 |
| 40° C./75% RH | | | |
| Stored 2 months at | 101.5 | 105.6 | 0.0354 |
| 40° C./75% RH | | | |
| Stored 1 month at | 99.9 | 107.9 | 0.079 |
| 50° C. | | | |
| Stored 1 month at | 101.1 | 108.4 | 0.0872 |
| 60° C. | | | |

*Percentage of original content.
**Percentage of impurity present in the composition.

### EXAMPLE 4

### Granisetron Hydrochloride (0.1 mg/mL, Single Dose) Injection Composition

| Ingredient | Grams/Liter |
|---|---|
| Granisetron hydrochloride | 0.11 |
| Citric acid anhydrous | 2 |
| Sodium chloride | 9 |
| Sodium hydroxide | q.s. to pH 5 ± 0.1 |
| (1N aqueous solution) | |
| Hydrochloric acid | q.s. to pH 5 ± 0.1 |
| (1N aqueous solution) | |
| Water for injection | q.s. to 1 liter |

Manufacturing process was similar to that described in Example 1.

The composition was subjected to a stability study involving storage for three months at “room” temperature (25° C./60% RH) and under accelerated stability conditions (40° C./75% RH), and the impurity contents, as determined by HPLC, were compared with those of KYTRIL® Injection 0.1 mg/mL single dose vials, similarly stored. Impurity analysis results are shown in the following table.

| | | IMPURITY (wt. %) | | | |
|---|---|---|---|---|---|
| SAMPLE | | A | C | Unknown | Total |
| KYTRIL ® | Initial | 0.07 | 0.01 | 0.01 | 0.11 |
| | 25° C./60% RH | ND | ND | 0.18 | 0.35 |
| | 40° C./75% RH | ND | ND | 0.18 | 0.4 |
| Example 4 | Initial | ND | 0.09 | ND | 0.1 |
| | 25° C./60% RH | ND | 0.01 | 0.08 | 0.2 |
| | 40° C./75% RH | ND | 0.08 | 0.05 | 0.1 |

ND: Not detected.

### EXAMPLE 5

### Granisetron Hydrochloride (1 mg/mL) Injection Composition with Meta-Cresol and Bronopol as Preservatives

| Ingredient | Grams/Liter |
|---|---|
| Granisetron hydrochloride | 0.11 |
| Citric acid anhydrous | 2 |
| Sodium chloride | 9 |
| Meta-cresol | 2.5 |
| Bronopol | 0.5 |
| Sodium hydroxide | q.s. to pH 5 ± 0.1 |
| (1N aqueous solution) | |
| Hydrochloric acid | q.s. to pH 5 ± 0.1 |
| (1N aqueous solution) | |
| Water for injection | q.s. to 1 liter |

Manufacturing process was similar to that described in Example 2, except for the use of meta-cresol and bronopol as preservatives in place of parabens.

### EXAMPLE 6

### Granisetron Hydrochloride 1 mg Tablets

| Ingredient | mg/Tablet |
|---|---|
| Granisetron hydrochloride | 1.12 |
| Lactose monohydrate (impalpable) | 73.1 |
| Microcrystalline cellulose | 10 |
| Hydoxypropyl methylcellulose (Hypromellose) (5 cps) | 2 |
| Water | 28 |
| Microcrystalline cellulose | 10 |
| Sodium starch glycolate | 3 |
| Magnesium stearate | 0.75 |
| Opadry White YS-1-7003 | 3.5 |
| Water | 84 |

Manufacturing Process:

1) Lactose monohydrate was sifted through a ASTM #40 mesh sieve and divided into three equal parts: A, B and C.

2) Granisetron hydrochloride, lactose monohydrate part A and microcrystalline cellulose PH101 were sifted through a ASTM #40 mesh sieve.

3) Presifted part B of lactose monohydrate and the step 2 ingredients were loaded into a double cone blender, followed by presifted part C of lactose monohydrate, and blended for 10 minutes.

4) The blend of step 3 was unloaded and sifted through a ASTM #40 mesh sieve.

5) The blend of step 4 was loaded into a double cone blender and blended for 10 minutes.

6) The blend of step 4 was unloaded and sifted through a ASTM #40 mesh sieve.

7) Hypromellose 5 cps was dispersed in hot water (50°-60° C.) while stirring slowly until a homogenous clear solution was formed.

8) The blend of step 6 was loaded into a rapid mixer granulator and granulated with the solution of step 7 and further was mixed for about 10 minutes.

9) Granulated mass from step 7 was dried in a fluid bed drier until the loss on drying was 1 to 2% w/w at 65±5° C.

10) The dried granules obtained in step 9 were sifted through a ASTM #24 mesh sieve and the retained particles were milled through a 1.5 mm screen fitted to a comminuting mill at medium speed, knives forward, and sifted through a ASTM #24 mesh sieve.

11) The granules from step 10 were blended with microcrystalline cellulose (Avicel PH102) and sodium starch glycolate, which was presifted through a ASTM #40 mesh sieve, in a double cone blender for about 15 minutes.

12) Magnesium stearate was sifted through a ASTM #60 mesh sieve, was added to the step 11 blend, and was further blended for about 5 minutes.

13) The lubricated blend of step 12 was compressed into tablets.

14) Opadry White YS-1-7003 was dispersed in water and stirred for 45 minutes to get a smooth homogenous suspension.

15) The tablets from step 13 were coated in automated coating equipment Neocota 24A until a weight gain of 3±0.5% was obtained.

A comparative stability study of granisetron HCl tablets with KYTRIL® was performed for a storage period of three months and the impurity content was analyzed by HPLC, giving the results below. The tablets from step 15 of Example 6 were divided into two lots for the test:

a) Lot A packaged in PVC/PVDC (60 gsm) blisters—Example 6a.

b) Lot B packaged in PVC/PVDC (120 gsm) blisters—Example 6b.

| | | IMPURITY (wt. %) | | | | |
|---|---|---|---|---|---|---|
| SAMPLE | | A | B | C | Unknown | Total |
| KYTRIL ® | Initial | 0.25 | 0.29 | 0.08 | 0.03 | — |
| | 25° C./60% RH | 0.28 | 0.25 | 0.05 | 0.04 | 0.68 |
| | 40° C./75% RH | 0.19 | 0.24 | 0.13 | 0.06 | 0.7 |
| Example 6a | Initial | ND | ND | 0.09 | 0.01 | 0.15 |
| | 25° C./60% RH | ND | ND | 0.10 | 0.02 | 0.13 |
| | 40° C./75% RH | ND | ND | 0.19 | 0.01 | 0.22 |
| Example 6b | Initial | ND | ND | 0.09 | 0.12 | 0.15 |
| | 25° C./60% RH | ND | ND | 0.10 | 0.18 | 0.13 |
| | 40° C./75% RH | ND | ND | 0.15 | 0.02 | 0.1 |

ND: Not detected.

The tablets of step 15 and KYTRIL® 1 mg tablets were subjected to dissolution testing using the following conditions:

Dissolution parameters:

Medium: 6.5 pH phosphate buffer.

Agitation: 50 RPM.

Apparatus: USP type II apparatus.

Volume: 500 mL.

### Drug release results are shown in the following table.

| | Cumulative % Granisetron Dissolved | | |
|---|---|---|---|
| Time | KYTRIL ® | | |
| (minutes) | 1 mg | Example 6a | Example 6b |
| 10 | 96 | 99 | 99 |
| 20 | 99 | 100 | 101 |
| 30 | 100 | 94 | 87 |
| 45 | 100 | 97 | 92 |
| 60 | 101 | 99 | 99 |

We claim:

1. An aqueous pharmaceutical composition comprising granisetron or a salt thereof and one or more paraben preservatives, the composition being sterilized by exposure to heat.

2. The aqueous pharmaceutical composition of claim 1, being substantially free from benzyl alcohol.

3. The aqueous pharmaceutical composition of claim 1, having a paraben preservative content about 0.01 percent to about 1 percent by weight.

4. The aqueous pharmaceutical composition of claim 1, having a paraben preservative content about 0.05 percent to about 0.5 percent by weight.

5. The aqueous pharmaceutical composition of claim 1, having a paraben preservative content about 0.1 percent to about 0.3 percent by weight.

6. The aqueous pharmaceutical composition of claim 1, wherein a paraben preservative comprises methyl paraben, present at about 0.01 percent to about 1 percent by weight.

7. The aqueous pharmaceutical composition of claim 1, wherein a paraben preservative comprises methyl paraben, present at about 0.05 percent to about 0.5 percent by weight.

8. The pharmaceutical composition of claim 1, wherein a paraben preservative comprises propyl paraben, present at about 0.01 percent to about 0.5 percent by weight.

9. The pharmaceutical composition of claim 1, wherein a paraben preservative comprises propyl paraben, present at about 0.01 percent to about 0.25 percent by weight.

10. The pharmaceutical composition of claim 1, wherein a paraben preservative comprises a ratio of methyl paraben to propyl paraben about 25:1 to 1:1, by weight.

11. The pharmaceutical composition of claim 1, wherein a paraben preservative comprises a ratio of methyl paraben to propyl paraben about 15:1 to 5:1, by weight.

12. The pharmaceutical composition of claim 1, having a 4-hydroxybenzoic acid content not more than about 0.4 percent by weight.

13. The pharmaceutical composition of claim 1, having a 4-hydroxybenzoic acid content not more than about 0.1 percent by weight.

14. An aqueous pharmaceutical composition comprising a salt of granisetron and about 0.01 percent to about 1 percent by weight of one or more paraben preservatives, the composition being sterilized by exposure to heat.

15. The aqueous pharmaceutical composition of claim 14, being substantially free from benzyl alcohol.

16. The aqueous pharmaceutical composition of claim 14, wherein a paraben preservative comprises methyl paraben, present at about 0.01 percent to about 1 percent by weight.

17. The pharmaceutical composition of claim 14, wherein a paraben preservative comprises propyl paraben, present at about 0.01 percent to about 0.5 percent by weight.

18. The pharmaceutical composition of claim 14, wherein a paraben preservative comprises a ratio of methyl paraben to propyl paraben about 25:1 to 1:1, by weight.

19. The pharmaceutical composition of claim 14, wherein a paraben preservative comprises a ratio of methyl paraben to propyl paraben about 15:1 to 5:1, by weight.

20. The pharmaceutical composition of claim 14, having a 4-hydroxybenzoic acid content not more than about 0.4 percent by weight.

21. The pharmaceutical composition of claim 14, having a 4-hydroxybenzoic acid content not more than about 0.1 percent by weight.

22. An aqueous pharmaceutical composition comprising a salt of granisetron and about 0.01 percent to about 1 percent by weight of a mixture of methyl paraben and propyl paraben, the composition being sterilized by exposure to heat and having a 4-hydroxybenzoic acid content not more than about 0.1 percent by weight.
